(19) United States
(12) Patent Application Publication
(10) Pub. No.: US 2017/0007506 A1
(43) Pub. Date: Jan. 12, 2017

(54) DENTAL GLASS IONOMER CEMENT COMPOSITION

(71) Applicant: GC CORPORATION, Tokyo (JP)

(72) Inventors: Yusuke HOKII, Tokyo (JP); Ryosuke YOSHIMITSU, Tokyo (JP); Futoshi FUSEJIMA, Tokyo (JP)

(21) Appl. No.: 15/121,140

(22) PCT Filed: Feb. 24, 2015
(86) PCT No.: PCT/JP2015/055116

§ 371 Date: Aug. 24, 2016

(30) Foreign Application Priority Data
Feb. 25, 2014 (JP) 2014-034358

(51) Int. Cl. A61K 6/083; A61K 6/06

(52) U.S. Cl. CPC A61K 6/0835; A61K 6/0612

(57) ABSTRACT

Provided is a dental glass ionomer cement composition whose hardened cement has a high strength, despite not including a (meth)acrylate monomer. The dental glass ionomer cement composition includes a filler in which a compound(s) having a carboxyl group(s) is/are bound to a surface of an inorganic powder via a silicon atom, the composition not including a (meth)acrylate monomer.

### TECHNICAL FIELD

The present invention relates to dental glass ionomer cement compositions that do not include a (meth)acrylate monomer.

### BACKGROUND ART

In using dental glass ionomer cements, μ-β unsaturated carboxylic acid polymers such as polycarboxylic acid, and fluoroaluminosilicate glass powders are allowed to react to each other in the presence of water to be hardened. This kind of dental glass ionomer cements are widely used in dentistry, because they have excellent characteristics, for example very good biocompatibility, excellent aesthetic property of semi-transparent hardened body, excellent adhesion to tooth substrates such as enamel and dentine, and an anticariogenic effect by fluoride contained in glass.

On the other hand, when a strong stress is applied to a hardened body of dental glass ionomer cement, cracks are created from fine pores inside and from scars on the surface of the hardened cement, and the hardened cement can be destroyed. This is considered to be because: the matrix portion formed by the cross-linking of α-β unsaturated carboxylic acid polymers and metal ions eluted from fluoroaluminosilicate glass, and interfaces between the matrix portion and the glass filler portion are fragile; therefore, if a stress is concentrated to fine scars created on the hardened cement, cracks rapidly expand in the matrix portion or the interfaces between the matrix portion and glass filler portion, whereby the hardened cement is destroyed.

As such, in order to obtain a good physical strength of a dental glass ionomer cement composition, it is effective to disperse the stress by increasing the blending amount of the glass component. However, if the blending amount of glass is increased, it gets very difficult to mix the powder and liquid of the cement, and the fluidity of the mixture gets worse, which causes a problem in operability.

Alternatively, there is a way of making a resin-modified glass ionomer cement by blending a polymerizable component ((meth)acrylate monomer) and a polymerization initiator for adding strength and toughness to the cement. However, this way has problems, for example the resin-modified glass ionomer cement cannot be used if a patient has an allergy to (meth)acrylate compounds.

As a method for improving the strength of a hardened cement without blending a (meth)acrylate monomer, disclosed is a dental cement whose physical strength is improved by including a high-strength fibrous strips and a CPSA (CaO—P₂O₅—SiO₂—Al₂O₃) glass fiber fine powder (for example, see Patent Literatures 1 and 2). However, if this kind of fibrous filler is blended, the powder and liquid get difficult to be mixed, and there also arises a practical problem that the surface smoothness of the hardened cement gets degraded if the ends of the fibers are exposed from the surface due to abrasions and the like.

In addition, in order to make glass having a fiber form, complicated processes are needed such as: melting and clarifying the raw material batch under a high temperature condition in advance; and melting the batch again to stretch the batch while rolling up the batch in a fiber form, and facilities for the complicated processes are also needed (see Patent Literature 2).

### CITATION LIST

### Patent Literature

### Patent Literature 1: JP S59-161307 A

### Patent Literature 2: JP 2000-119119 A

### SUMMARY OF INVENTION

### Technical Problem

An object of the present invention is to provide a dental glass ionomer cement composition whose hardened cement has a high strength despite not including a (meth)acrylate monomer.

### Solution to Problem

As a result of intensive researches for solving the above problems, the inventors of the present invention found: it is possible to obtain a dental glass ionomer cement composition whose hardened cement has a high strength, by blending, with the composition, a filler in which a compound(s) having a carboxyl group(s) is/are bound to a surface of an inorganic powder via a silicon atom, despite not including a (meth)acrylate monomer in the composition; and this is because the filler stabilizes in the composition due to an interaction between the carboxyl group(s) and the matrix portion of the cement via metal ions. The present invention has been completed based on the above finding.

That is, the present invention is a dental glass ionomer cement composition including a filler in which compound(s) having a carboxyl group(s) is/are bound to a surface of an inorganic powder via a silicon atom, the dental glass ionomer cement composition not including a (meth)acrylate monomer.

Specifically, the present invention is a dental glass ionomer cement composition including:
a powder component whose main component is
   (A) a fluoroaluminosilicate glass powder; and
a liquid component whose main components are
   (B) an α-β unsaturated carboxylic acid polymer and
   (C) water,
      wherein:
at least one of the powder component and the liquid component include
   (D) a filler in which a compound(s) having a carboxyl group(s) is/are bound to a surface of an inorganic powder via a silicon atom; and
neither the powder component nor the liquid component includes a (meth)acrylate monomer.

Here, “main component” means the component that accounts more than 50 mass % (majority) of the composition. If one substance is given as the main component, this one substance accounts more than 50 mass % of the component, and if a plurality of substances are given, the sum of the given substances accounts more than 50 mass % of the composition.

Another embodiment of the present invention is a dental glass ionomer cement composition including:
a first paste whose main components are
   (A) a fluoroaluminosilicate glass powder and
   (C) water; and
a second paste whose main components are
   (B) an α-β unsaturated carboxylic acid polymer and
   (C) water,
      wherein:
the second paste includes
   (D) a filler in which a compound(s) having a carboxyl group(s) is/are bound to a surface of an inorganic powder via a silicon atom; and
neither the first paste nor the second paste includes a (meth)acrylate monomer.

### Advantageous Effects of Invention

The dental glass ionomer cement composition according to the present invention is a dental glass ionomer cement composition whose hardened cement has a high strength, despite not including a (meth)acrylate monomer.

### DESCRIPTION OF EMBODIMENTS

Hereinafter the embodiments of the present invention will be described in detail.

First, a powder-liquid type dental glass ionomer cement composition which is one embodiment of the dental glass ionomer cement composition of the present invention will be described. The powder-liquid type dental glass ionomer cement composition is a dental glass ionomer cement composition including:
a powder component whose main component is
   (A) a fluoroaluminosilicate glass powder; and
a liquid component whose main components are
   (B) an α-β unsaturated carboxylic acid polymer and
   (C) water,
      wherein:
at least one of the powder component and the liquid component include
   (D) a filler in which a compound(s) having a carboxyl group(s) is/are bound to a surface of an inorganic powder via a silicon atom; and
neither the powder component nor the liquid component includes a (meth)acrylate monomer. In the present invention, “(meth)acrylate” means “at least one of methacrylate and acrylate”.

The powder component of the powder-liquid type dental glass ionomer cement composition according to the first embodiment includes (A) a fluoroaluminosilicate glass powder. The fluoroaluminosilicate glass powder is preferably a fluoroaluminosilicate glass powder whose average particle size is in the range of from 0.02 μm to 10 μm, and whose specific gravity is in the range of from 2.4 to 4.0, including Al³⁺, Si⁴⁺, F⁻ and O²⁻ as main components, and further including at least one of Sr²⁺ and Ca²⁺. If the average particle size is more than 10 μm, the surface smoothness of the hardened cement is difficult to be obtained, therefore the contact feeling in an oral cavity gets worse. On the other hand, if a fine powder of less than 0.02 μm in average particle size is used, there is a possibility that the physical property degrades because it is difficult to include an absolute quantity of the powder. The particle size can be measured by a general method, and is shown by the average value of the major axis and minor axis.

The fluoroaluminosilicate glass powder (A) can be made by a known method of making glass. The blending amount of the fluoroaluminosilicate glass powder is preferably in the range of from 70 mass % to 100 mass % in the powder component. If the amount is less than 70 mass %, the physical property of the hardened cement tends to degrade.

The powder-liquid type dental glass ionomer cement composition according to the first embodiment includes (B) an α-β unsaturated carboxylic acid polymer, at least in the liquid component. It is preferable that the α-β unsaturated carboxylic acid polymer is a polymer of unsaturated monocarboxylic acid or a polymer of α-β unsaturated dicarboxylic acid. The α-β unsaturated carboxylic acid polymer is preferably a copolymer or a homopolymer including one or more kinds selected from acrylic acid, methacrylic acid, 2-chloroacrylic acid, 3-chloroacrylic acid, aconitic acid, mesaconic acid, maleic acid, itaconic acid, fumaric acid, glutaconic acid, and citraconic acid, not having a polymerizable unsaturated double bond, and whose mass average molecular weight is in the range of from 5000 to 40000. If a polymer whose mass average molecular weight is less than 5000 is used for these α-β unsaturated carboxylic acids, the strength of the cement after hardened tends to degrade, and the adhesion strength to tooth substances also tends to degrade. If a polymer whose mass average molecular weight is more than 40000 is used, the viscosity in mixing gets large and the mixing tends to be difficult.

The blending amount of the α-β unsaturated carboxylic acid (B) is preferably in the range of from 20 mass % to 50 mass % in the liquid component. If the amount is less than 20 mass %, the strength of the hardened cement tends to degrade, and if the amount is more than 50 mass %, the mixing tends to be difficult. In addition, the blending amount in the powder component is preferably in the range of from 0 mass % to 20 mass %. If the amount is more than 20 mass %, the physical property of the hardened cement tends to degrade.

The powder-liquid type dental glass ionomer cement composition according to the first embodiment includes (C) water in the liquid component. Water is an essential component in the present invention. This is because the neutralizing reaction of the fluoroaluminosilicate glass (A) and the α-β unsaturated carboxylic acid (B) progresses under the existence of water. In addition, the powder-liquid type dental glass ionomer cement composition according to the first embodiment adheres to surfaces of teeth under the existence of water.

The blending amount of (C) water is preferably in the range of from 40 mass % to 80 mass % in the liquid component. If the amount is less than 40 mass %, the mixing tends to be difficult, and if the amount is more than 80 mass %, the strength of the hardened cement tends to degrade.

The powder-liquid type dental glass ionomer cement composition according to the first embodiment includes (D) a filler in which a compound(s) having a carboxyl group(s) is/are bound to a surface of an inorganic powder via a silicon atom, in at least one of the powder component and the liquid component. Because of this, the filler stabilizes in the component due to the interaction between the carboxyl group (s) and the matrix portion of the cement via metal ions. Therefore it is possible to obtain a dental glass ionomer cement composition whose hardened body has a high strength, despite not including a (meth)acrylate monomer.

The filler (D) in which a compound(s) having a carboxyl group(s) is/are bound to a surface of an inorganic powder via a silicon atom is manufactured by bonding:

(a) an inorganic powder whose surface is treated by a silane coupling agent having an unsaturated double bond(s); and
(b) carboxylic acid(s) having an unsaturated double bond(s) exclusive of a (meth)acrylate compound.

Examples of the inorganic powder (a) include: colloidal silica that does not react with acids under the existence of water; silica powder such as crystalline silica; silica sand which is a mineral; quarts; crystalline glasses that do not emit metal ions such as strontium glass, barium glass, and borosilicate glass; fluoroaluminosilicate glass that has reactivity with acids under the existence of water; alumina powder; titanium oxide powder; and barium sulfate. If water is not used for (d) a dispersion medium which is described later, a mixture of two or more kinds of these inorganic powders can be used. If water is used for the dispersion medium (d), a mixture of two or more kinds of the inorganic powders that have the same reactivity with water can be used. Among them, one or two or more kinds selected from the group consisting of silica powder, quarts, alumina powder, titanium oxide powder, and fluorialuminosilicate glass are preferable.

The average particle size of the inorganic powder (a) is preferably in the range of from 0.02 μm to 10 μm. If the average particle size is more than 10 μm, the surface smoothness of the cement after hardened is not obtained, therefore the contact feeling in an oral cavity gets worse. On the other hand, if a fine powder of less than 0.02 μm in average particle size is used, it gets difficult to mix an absolute quantity of the inorganic powder in the composition in use, therefore the physical property of the hardened cement tends to degrade.

The surface of the inorganic powder (a) needs to be treated with a silane coupling agent having an unsaturated double bond(s). Examples of the silane coupling agent having an unsaturated double bond(s) used for the surface treatment include vinyl-based silane coupling agents such as vinyltrimethoxysilane, vinyltriethoxysilane, γ-methacryloxypropyltrimethoxysilane, γ-methacryloxypropylmethyldimethoxysilane, vinyltrichlorosilane, and vinyltris(2-methoxyethoxy)silane. This treatment chemically fixes the silane coupling agent having an unsaturated double bond(s) on the surface of the inorganic powder. A carbon atom on the unsaturated double bond binds with a carbon atom on the unsaturated double bond of the carboxylic acid (b) having an unsaturated double bond(s) exclusive of a (meth)acrylate compound.

The used amount of the silane coupling agent is, to 100 pts. mass of the inorganic powder, preferably in the range of from 0.01 pts. mass to 20 pts. mass. If the amount is less than 0.01 pts. mass, a sufficient strength tends not to be obtained, and if the amount is more than 20 pts. mass, a uniform treatment powder tends not to be obtained.

The blending amount of the inorganic powder (a) whose surface is treated with a silane coupling agent having an unsaturated double bond(s) is preferably in the range of from 20 mass % to 80 mass %, to the total amount of the inorganic powder (a), the carboxylic acid (b) and (c) a polymerization initiator which is described later. If the amount is less than 20 mass %, the effect is difficult to be obtained, and if the amount is more than 80 mass %, the preparation itself of the reaction liquid tends to be difficult.

Regarding the carboxylic acid (b) having an unsaturated double bond(s) exclusive of a (meth)acrylate compound, a carbon atom on the unsaturated double bond binds with a carbon atom on the unsaturated double bond of the silane coupling agent which is bound to the surface of the inorganic powder described above. This eventually introduces the carboxyl group to the surface of the filler (D) via a silicon atom, and the strength of the filler itself improves. Examples of the carboxylic acid having an unsaturated double bond(s) exclusive of a (meth)acrylate compound include acrylic acid, methacrylic acid, itaconic acid, fumaric acid, maleic acid, aconitic acid, angelic acid, citraconic acid, crotonic acid, glutaconic acid, metaconic acid, metaconic acid, muconic acid, tiglic acid, cinnamic acid, allylmalonic acid, butenoic acid, pentenoic acid, hexenoic acid, heptenoic acid, octenoic acid, nonenoic acid, decenoic acid, oleic acid, linoleic acid, and linolenic acid. The acid anhydrides thereof, structural isomers thereof, and cis-trans isomers thereof are also included in the examples. These may be used alone, and a mixture of two or more kinds thereof may also be used. “exclusive of a (meth)acrylate compound” means that the carboxylic acid does not have the partial structure shown by the following chemical formula (X is a hydrogen atom or methyl group, and the structure beyond the dashed line may be in any form).

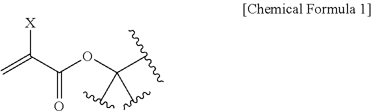

Regarding the carboxylic acid (b), the smaller molecular weight the carboxylic acid has, the better, because the number of carboxyl groups in the molecular gets relatively large. A preferable range of the molecular weight of the carboxylic acid is from 70 to 175, and in the above examples of the carboxylic acid, acrylic acid, methacrylic acid, itaconic acid, fumaric acid, maleic acid, aconitic acid, angelic acid, citraconic acid, crotonic acid, glutaconic acid, metaconic acid, mesaconic acid, muconic acid, tiglic acid, cinnamic acid, allylmalonic acid, butenoic acid, pentenoic acid, hexenoic acid, heptenoic acid octenoic acid, nonenoic acid, and decenoic acid fall under the range. Further preferable range is from 70 to 135, and in the examples of the carboxylic acid, acrylic acid, methacrylic acid, itaconic acid, fumaric acid, maleic acid, angelic acid, citraconic acid, crotonic acid, glutaconic acid, metaconic acid, mesaconic acid, tiglic acid, butenoic acid, pentenoic acid, hexenoic acid, and heptenoic acid fall under the range.

In addition, a carboxylic acid having a plurality of carboxyl groups in one molecule is preferable because the number of the carboxyl groups in the molecule gets relatively large. In the examples of the carboxylic acid, itaconic acid, fumaric acid, maleic acid, aconitic acid, citraconic acid, glutaconic acid, metaconic acid, mesaconic acid, muconic acid, and allylmalonic acid fall into the preferable carboxylic acid.

In addition, it is preferable that the carboxylic acid (b) does not include nitrogen atom, phosphorus atom, sulfur atom, or the like, considering safety as a dental material etc. That is, the carboxylic acid (b) is preferably a carboxylic acid constituted only by three kinds of atoms of hydrogen, carbon, and oxygen.

Putting the above together, as the carboxylic acid (b), acrylic acid, itaconic acid, maleic acid, metaconic acid, and mesaconic acid are especially preferable, which have excellent effects to improve the strength of the hardened cement.

On the other hand, the polyacids formed from polymerized carboxylic acid (b) cannot be used because the unsaturated double bond(s) of them are lost by the polymerization.

In the present invention, the step of bonding the inorganic powder (a) and the carboxylic acid (b) is carried out by liquid-phase polymerization or emulsion polymerization. That is, the reaction of the inorganic powder (a), the carboxylic acid (b), and an appropriate polymerization initiator (c) is carried out in an appropriate dispersion medium (d). According to this method, it is possible to bond the carboxylic acid (b) homogeneously and highly densely, because the inorganic powder (a) is dispersed in the dispersion medium. As such, it is possible to obtain a homogeneous and highly effective filler. In addition, it has an advantage of very easy manufacturing, because purification can be done only by removing the dispersion medium after the polymerization. In contrast, solid-phase polymerization which is a conventional and general making method of organic-inorganic composition fillers does not have the above-described advantage, therefore it is difficult to obtain a homogeneous and highly-effective filler. In addition, with solid-phase polymerization, a grinding and particle size adjustment are needed after the polymerization, which causes the manufacturing cost to increase. Therefore, solid-phase polymerization is not applied in the present invention.

The blending amount of the carboxylic acid (b) having an unsaturated double bond(s) exclusive of a (meth)acrylate compound is preferably in the range of from 20 mass % to 80 mass % to the total amount of (a) to (c). If the amount is less than 20 mass %, the effect is difficult to be obtained, and if the amount is more than 80 mass %, the preparation itself of the reaction liquid tends to be difficult.

The polymerization initiator (c) has a function to bind the carboxylic acid (b) having an unsaturated double bond(s) exclusive of a (met)acrylate compound and the inorganic powder (a) treated with a silane coupling agent having an unsaturated double bond(s) between their unsaturated double bonds, via carbon-carbon single bond(s). As the polymerization initiator, polymerization initiators used for conventional dental materials can be used without particular limitations, and an effective method for polymerization reaction system can be adequately chosen depending on the kind and combination of the polymerization initiator. Specifically, thermal polymerization initiators are preferable for the purpose of securing the polymerization. As the thermal polymerization initiator, organic metal compounds such as azobisisobutyronitrile as an azo compound and tributyl borate are preferable. Diacyl peroxides having aromatic rings, and peroxy esters that are regarded as esters of perbenzoic acids, such as benzoyl peroxide, 2,4-dichlorobenzoyl peroxide, m-tolyl peroxide, t-butyl peroxybenzoate, di-t-butyl peroxyisophthalate, 2,5-dimethyl-2,5di(benzoylperoxy)hexane, and 2,5-dimethyl-2,5di[(o-benzoyl)benzoylperoxy]hexane can be used. As a water-soluble polymerization initiator, persulfates such as potassium peroxodisulfate, sodium peroxodisulfate, ammonium persulfate, potassium persulfate, sodium persulfate, potassium peroxydiphosphate, and potassium peroxydisulfate are also preferable. These polymerization initiators may be used alone, and a mixture of two or more kinds thereof may also be used. If a polymerization initiator that is insoluble in alcohol, such as potassium peroxodisulfate, is used, a dispersion medium that dissolves the initiator needs to be used.

The blending amount of the polymerization initiator (c) is preferably in the range of from 0.1 mass % to 3 mass % to the total amount of (a) to (c). If the amount is less than 0.1 mass %, it is difficult to obtain a sufficient polymerization effect. On the other hand, though depending on the kind and blending ratio of the unsaturated carboxylic acid to be used, if the amount is more than 3 mass %, the length of the polymer chain to be obtained gets short, and a sufficient effect on improvement in the physical property of the cement tends not to be obtained.

As the dispersion medium (d), a medium that sufficiently disperses the inorganic powder treated with a silane coupling agent having an unsaturated double bond(s), and that sufficiently dissolves both the carboxylic acid (b) and the polymerization initiator (c), is preferably used. A mixture solvent of water and alcohol is especially preferably used. In specific, the alcohol concentration in the mixture solvent is preferably in the range of from 30% to 80%. If the concentration is more than 80%, the solubility of the water-soluble polymerization initiator tends to degrade, whereby the reaction efficiency tends to degrade. If the concentration is less than 30%, the dispersion efficiency of the inorganic powder (a) treated with a silane coupling agent having an unsaturated double bond(s) tends to degrade.

If an inorganic powder having reactivity with acids is used for the inorganic powder (a), a non-aqueous dispersion medium needs to be selected for the dispersion medium (d), in order to prevent the progression of acid-base reaction in the polymerization reaction. Considering the solubility of the carboxylic acid (b), a lower alcohol is preferably used, and ethanol is especially preferably used. It is preferable to carry out a dehydration treatment on the dispersion medium by molecular sieves before the medium is used.

By a polymerization reaction of a mixture including the above (a) to (c), the carboxylic acid (b) having an unsaturated double bond(s) chemically binds with the inorganic powder (a) treated with a silane coupling agent having an unsaturated double bond(s), via a silicon atom. That is, a carbon atom on the unsaturated double bond in an extension of the silicon atom of the inorganic powder (a) treated with a silane coupling agent having an unsaturated double bond(s) and a carbon atom on the unsaturated double bond of the carboxylic acid (b) having an unsaturated double bond(s) chemically bind to each other. This makes it possible to obtain a filler in which a compound(s) having a carboxyl group(s) is/are bound to a surface of an inorganic powder via a silicon atom. An effective method for polymerization reaction system can be adequately chosen depending on the kind and combination of the polymerization initiator.

The filler (D) can of course include an antibacterial agent, colorant, stabilizer, and the like which are normally used, if necessary.

The filler (D) in which a compound (s) having a carboxyl group(s) is/are bound on a surface of an inorganic powder via a silicon atom is blended in at least one of the powder component and the liquid component of the dental glass ionomer cement composition according to the present invention. It should be noted that the filler (D) cannot be blended in the liquid component if an inorganic powder that has reactivity with the α-β unsaturated carboxylic acid polymer is used as a raw material of the filler (D). When the filler (D) is blended in the powder component, the amount is preferably in the range of from 0.1 mass % to 10 mass %. If the amount is less than 0.1 mass %, the interaction of providing a sufficient strength between the matrix portion of the cement and the inorganic powder, after the cement is hardened, does not tend to be obtained. If the amount is more than 10 mass %, the operability tends to be seriously impaired. The amount is more preferably in the range of from 1 mass % to 5 mass %.

When the filler is blended in the liquid component, the amount is preferably in the range of from 1 mass % to 10 mass %. If the amount is less than 1 mass %, the interaction of providing a sufficient strength between the matrix portion of the cement and the inorganic powder, after the cement is hardened, does not tend to be obtained. If the amount is more than 10 mass %, the operability tends to be seriously impaired and the mixing gets difficult. The amount is more preferably in the range of from 1 mass % to 5 mass %.

The powder-liquid type dental glass ionomer cement composition according to the first embodiment may include, if necessary, (E) an acid, in the liquid component, for adjusting pH. Examples of the acid to be used include phosphoric acid, citric acid, succinic acid, oxalic acid, fumaric acid, tartaric acid, malic acid, maleic acid, ethylenediaminetetraacetic acid, tricarballylic acid, levulinic acid, acidic amino acid, pyroglutamic acid, L-aspartic acid, L-arginine, citric acid, glycine, glycolic acid, DL-glyceric acid, gluconic acid, glucuronic acid, glutaric acid, acetone dicarboxylic acid, cyclopentane tetracarboxylic acid, diglycolic acid, diethylmalonic acid, L-cysteic acid, oxalic acid, sulfosalicylic acid, tartronic acid, tricarballylic acid, tetrahydrofurantetracarboxylic acid, meso-butane-1,2,3,4-tetracarboxylic acid, trimellitic acid, lactic acid, benzenepentacarboxylic acid, malonic acid, DL-mandelic acid, benzenhexacarboxylic acid, and malic acid. These acids may be used alone, and a mixture of two or more kinds thereof may also be used.

The blending amount of the acid (E) in the liquid component is preferably in the range of from 0.5 mass % to 20 mass %. If the amount is less than 0.5 mass %, the effect is hardly obtained, and if the amount is more than 20 mass %, there is a possibility that the bending strength of the hardened cement degrades.

Next, a paste type dental glass ionomer cement composition which is another embodiment of the dental glass ionomer cement composition according to the present invention will be described. The paste type dental glass ionomer cement composition is a dental glass ionomer cement composition including:
a first paste whose main components are
   (A) a fluoroaluminosilicate glass powder and
   (C) water; and
a second paste whose main components are
   (B) an α-β unsaturated carboxylic acid polymer and
   (C) water,
      wherein:
the second paste includes
   (D) a filler in which a compound(s) having a carboxyl group(s) is/are bound to a surface of an inorganic powder via a silicon atom; and
neither the first paste nor the second paste includes a (meth)acrylate monomer.

The paste type dental glass ionomer cement composition according to the second embodiment includes (A) a fluoroaluminosilicate glass powder in the first paste. As the fluoroaluminosilicate glass powder, powders that can be used in the above embodiment of powder-liquid type can be used in the same way.

The blending amount of the fluoroaluminosilicate glass powder (A) is preferably in the range of from 40 mass % to 85 mass % in the first paste. If the amount is less than 40 mass %, the physical property of the hardened cement may degrade, and if the amount is more than 85 mass %, the second paste gets hard and the operability in mixing tends to get worse.

The paste type dental glass ionomer cement composition according to the second embodiment includes (B) an α-β unsaturated carboxylic acid polymer in the second paste. As the α-β unsaturated carboxylic acid polymer, the polymers that can be used in the above embodiment of powder-liquid type can be used.

The blending amount of the α-β unsaturated carboxylic acid polymer (B) is preferably in the range of from 20 mass % to 60 mass % in the second paste. If the amount is less than 20 mass %, the adhesion property to tooth substances, which is one characteristic of the dental glass ionomer cement, easily degrades, and if the amount is more than 60 mass %, the preparation of the paste tends to be difficult.

The paste type dental glass ionomer cement composition according to the second embodiment includes (C) water, in both the first and second pastes. Water is an essential component in the present invention. This is because the neutralizing reaction of the fluoroaluminosilicate glass (A) and the α-β unsaturated carboxylic acid polymer (B) progresses under the existence of water. In addition, the paste type dental glass ionomer cement composition according to the second embodiment adheres to the surfaces of teeth under the existence of water.

The blending amount of (C) water is preferably in the range of from 10 mass % to 40 mass % in the first paste. If the amount is less than 10 mass %, the adhesion to tooth substances which is a characteristic of the dental glass ionomer cement degrades, and if the amount is more than 40 mass %, the physical property after the cement is hardened tends to degrade. The blending amount of (C) water in the second paste is preferably in the range of from 20 mass % to 60 mass %. If the amount is less than 20 mass %, the adhesion to tooth substrates which is a characteristic of the dental glass ionomer cement degrades, and if the amount is more than 60 mass %, the physical property after the cement is hardened tends to degrade.

Part of (C) water in the first paste may be replaced by (F) glycerin, for the purpose of adjusting the fluidity of the paste.

The blending amount of (F) glycerin is preferably in the range of from 2 mass % to 20 mass % in the first paste. If the amount is less than 2 mass %, the effect is hardly obtained, and if the amount is more than 20 mass %, the first paste gets hard and the operability in mixing tends to get worse easily.

The paste type dental glass ionomer cement composition according to the second embodiment includes, at least in the second paste, (D) a filler in which a compound(s) having a carboxyl group(s) is/are bound to a surface of an inorganic powder via a silicon atom. This makes the filler stabilize in the composition by the interaction of the carboxyl group(s) and the matrix portion of the cement via metal ions, and thereby it is possible to obtain a dental glass ionomer cement composition whose hardened cement has a high strength, despite not including a (meth)acrylate monomer. As the filler, the same fillers described in the above embodiment of the powder-liquid type can be used in the same way. However, the filler (D) cannot be blended in the second paste if an inorganic powder that has reactivity with the α-β unsaturated carboxylic acid polymer (B) is used as a raw material of the filler (D).

The blending amount of the filler (D) in which a compound(s) having a carboxyl group(s) is/are bound to a surface of an inorganic powder via a silicon atom is preferably in the range of from 5 mass % to 40 mass % in the second paste. If the amount is less than 5 mass %, the interaction of providing a sufficient strength between the matrix portion of the cement and the inorganic powder, after the cement is hardened, does not tend to be obtained. If the amount is more than 40 mass %, the operability tends to be seriously impaired. When the filler is blended in the first paste, the amount of the filler in the first paste is preferably in the range of from 0 mass % to 20 mass %. If the amount is more than 20 mass %, the hardenability tends to degrade.

The paste type dental glass ionomer cement composition according to the second embodiment may include (G) a thickening agent in the first paste, for the purpose of adjusting the operability. As the thickening agent, both of inorganic thickening agents and organic thickening agents may be used. Examples of the thickening agent include fine particle silica, calcium carboxymethyl cellulose, sodium carboxymethyl cellulose, starch, sodium starch glycolate, sodium starch phosphate, methylcellulose, sodium polyacrylate, alginic acid, sodium alginate, propylene glycol alginate, casein, sodium caseinate, polyethylene glycol, ethyl cellulose, hydroxyethyl cellulose, gluten, locust bean gum, and gelatin. Among them, calcium carboxymethyl cellulose and sodium carboxymethyl cellulose are preferable because they have high thickening effects with small amounts and are inexpensive. A mixture of two or more kinds of these thickening agents can of course be used.

The blending amount of the thickening agent (G) is preferably in the range of from 0.005 mass % to 2 mass % in the first paste. If the amount is less than 0.005 mass %, the effect is hardly obtained, and if the amount is more than 2 mass %, the physical property of the hardened cement tends to degrade. The amount is more preferably in the range of from 0.005 mass % to 0.4 mass %.

The paste type dental glass ionomer cement composition according to the second embodiment can of course include an antibacterial agent, colorant, stabilizer, and the like which are normally used, if necessary.

Hereinafter the dental glass ionomer cement composition according to the present invention will be specifically described with examples. However, the present invention is not limited to these examples.

### Examples

### Preparation of Filler

Fillers 1 to 11 to be blended in the dental glass ionomer cement compositions of Examples and Comparative Examples were prepared. Each of the fillers 1 to 8 was the filler (D) in which a compound(s) having a carboxyl group(s) is/are bound to a surface of an inorganic powder via a silicon atom. The Fillers 9 to 11 were other fillers used in Comparative Examples.

### Filler 1

γ-methacryloxypropyltrimethoxysilane in an amount of 20 pts. mass diluted with ethanol to be 50 mass % was added to 100 pts. mass of quartz powder whose average particle size was 1.8 μm, and mixed by an automatic grinding machine. The obtained powder was subjected to a heat treatment at 110° C. for 2 hours. The obtained treatment powder was determined as a quartz powder 10% silane-treated inorganic powder.

Next, a mixed liquid of water:ethanol=2:1 was prepared to be (d) a dispersion medium. The silane-treated inorganic powder in an amount of 45.3 mass % and itaconic acid in an amount of 53.5 mass %, to the total amount of (a) to (c), were put in a reaction container, and thereafter diluted in a measuring cylinder by the mixed liquid so that the resultant liquid was 100 ml. The resultant liquid was stirred at 60° C. for 30 minutes. After that, 1.2 mass % of potassium peroxodisulfate was put in the liquid, and polymerized for 1 hour. A centrifugal separation was carried out to the obtained liquid to remove supernatant. Thereafter, the resultant precipitation was suspended again in water. This operation was repeated for four times, to remove unreacted itaconic acid and polymerization initiator. Further, moisture was removed from the precipitation by a freeze dryer, whereby a white filler (D) in a bulky powder form in which itaconic acid was bound to a surface of the quartz powder via a silicon atom was obtained.

### Fillers 2 to 5

The fillers 2 to 5 were prepared in the same way as in preparing the filler 1.

### Fillers 6 to 8

The fillers 6 to 8 were prepared in the same way as in preparing the filler 1, except that a fluoroaluminosilicate glass powder 2% silane treated inorganic powder was used as the inorganic powder (a), azobisisobutyronitrile was used as the polymerization initiator (c), and ethanol to which a dehydration treatment was carried out by molecular sieves was used as the dispersion medium (d).

### Filler 9

A quarts powder was used as it was as the filler 9.

### Filler 10

The filler 10 was prepared in the same way as in preparing the filler 1, except that the polymerization initiator (c) was not included therein.

### Filler 11

The filler 11 was prepared in the same way as in preparing the filler 1, except that ethanol to which a dehydration treatment was carried out by molecular sieves was used as the dispersion medium (d). Each blending amount and the average particle size of the obtained fillers are shown together in Table 1.

TABLE 1

| | | Fillers used in Examples | | | | | |
|---|---|---|---|---|---|---|---|
| | | Filler 1 | Filler 2 | Filler 3 | Filler 4 | Filler 5 | Filler 6 |
| (a) Inorganic powder whose | Quarts powder 10% silane | 45.3 | 45.3 | 45.3 | 45.3 | 45.3 | |
| surface is treated with silane | treated inorganic powder | | | | | | |
| treatment agent having | Fluoroaluminosilicate glass | | | | | | 56.9 |
| unsaturated double bond(s) | powder A 2% silane treated | | | | | | |
| (mass %) | inorganic powder | | | | | | |
| | Fluoroaluminosilicate glass | | | | | | |
| | powder B 2% silane treated | | | | | | |
| | inorganic powder | | | | | | |
| Other powder | Quarts powder | | | | | | |
| (b) Carboxylic acid having | Itaconic acid | 53.5 | 53.5 | 53.5 | 44.4 | 44.4 | 42.6 |
| unsaturated double bond(s) | Acrylic acid | | | | 9.1 | | |
| exclusive of (meth)acrylate | Maleic acid | | | | | 9.1 | |
| compound (mass %) | | | | | | | |
| (c) Polymerization initiator | Potassium peroxodisulfate | 1.2 | 1.2 | 1.2 | 1.2 | 1.2 | |
| (mass %) | Azobisisobutyronitrile | | | | | | 0.5 |

| | | | | | | |
|---|---|---|---|---|---|---|
| (d) Dispersion medium (water:ethanol) or only ethanol | 2:1 | 2:1 | 2:1 | 2:1 | 2:1 | ethanol |
| | | | | | | only |
| Reaction condition | 60° C. | 60° C. | 60° C. | 60° C. | 60° C. | 60° C. |
| | 1 hour | 3 hour | 6 hour | 6 hour | 6 hour | 6 hour |
| Average particle size (μm) | 1.8 | 1.8 | 1.8 | 1.8 | 1.8 | 2.4 |
| Zeta potential (mV) | −19 | −35 | −38 | −43 | −35 | −41 |

| | | Fillers used | | Fillers used in | | |
|---|---|---|---|---|---|---|
| | | in Examples | | Comparative Examples | | |
| | | Filler 7 | Filler 8 | Filler 9 | Filler 10 | Filler 11 |
| (a) Inorganic powder whose | Quarts powder 10% silane | | | | 45.8 | 45.3 |
| surface is treated with silane | treated inorganic powder | | | | | |
| treatment agent having | Fluoroaluminosilicate glass | 60.3 | | | | |
| unsaturated double bond(s) | powder A 2% silane treated | | | | | |
| (mass %) | inorganic powder | | | | | |
| | Fluoroaluminosilicate glass | | 60.3 | | | |
| | powder B 2% silane treated | | | | | |
| | inorganic powder | | | | | |
| Other powder | Quarts powder | | | 100.0 | | |
| (b) Carboxylic acid having | Itaconic acid | 30.2 | 30.2 | | 54.2 | 53.5 |
| unsaturated double bond(s) | Acrylic acid | 9.0 | | | | |
| exclusive of (meth)acrylate | Maleic acid | | 90 | | | |
| compound (mass %) | | | | | | |
| (c) Polymerization initiator | Potassium peroxodisulfate | | | | | 1.2 |
| (mass %) | Azobisisobutyronitrile | 0.5 | 0.5 | | | |

| | | | | | |
|---|---|---|---|---|---|
| (d) Dispersion medium (water:ethanol) or only ethanol | ethanol | ethanol | | 2:1 | ethanol |
| | only | only | | | only |
| Reaction condition | 60° C. | 60° C. | | 60° C. | 60° C. |
| | 6 hour | 6 hour | | 3 hour | 6 hour |
| Average particle size (μm) | 2.4 | 2.4 | 1.8 | 1.8 | 1.8 |
| Zeta potential (mV) | −40 | −38 | −16 | −14 | −14 |

### Preparation of Fluoroaluminosilicate Glass Powder

### Fluoroaluminosilicate Glass Powder a

By a mortar, 22 g of aluminum oxide, 23 g of silica, 12 g of calcium fluoride, 15 g of calcium phosphate, and 28 g of strontium fluoride were sufficiently mixed and stirred. The obtained batch was put in a porcelain crucible and heated to 1200° C. at approximately 7° C./min of temperature rising rate by an electric furnace, and kept at 1200° C. for 3 hours. The obtained melt was poured into water, whereby a quenched glass was obtained. The quenched glass was pulverized to be a fluoroaluminosilicate glass powder A. The average particle size of this inorganic powder was 2.5 μm.

### Fluoroaluminosilicate Glass Powder B

By a mortar, 23 g of aluminum oxide, 31 g of silica, 1 g of calcium fluoride, 9 g of cryolite, 2 g of aluminum phosphate, and 34 g of strontium fluoride were sufficiently mixed and stirred. The obtained batch was put in a porcelain crucible and heated to 1200° C. at approximately 7° C./min of temperature rising rate by an electric furnace, and kept at 1200° C. for 3 hours. The obtained melt was poured into water, whereby a quenched glass was obtained. The quenched glass was pulverized to be a fluoroaluminosilicate glass powder B. The average particle size of this inorganic powder was 2.5 μm.

<<Evaluation of Filler by Zeta Electrometer>>

The obtained filler was suspended in 10 mM Nacl-0.01% TritonX-100 solution, so that the amount of the filler was 0.1 mass % in the solution. The resultant suspension was irradiated with ultrasonic waves for 10 minutes, whereby the filler was sufficiently dispersed. This suspension was used as a measurement specimen. The variation of electric charge on the surface of the specimen was measured by a zeta electrometer (ELS-Z, manufactured by OTSUKA ELECTRONICS CO., LTD.). The bonding amount of carboxyl groups was relatively larger as the value of the zeta potential was negative and its absolute value was larger. The results are together shown in Table 1.

<<Making of Powder-Liquid Type Dental Glass Ionomer Cement Composition>>

Each component was mixed at the blending amount shown in Table 2, whereby powder components and liquid components of powder-liquid type dental glass ionomer cement compositions were prepared.

<<Bending Strength Test>>

Each powder component and liquid component prepared above was scaled at the powder-liquid ratio shown in Table 2. The scaled components were mixed for 30 seconds on a mixing paper by a spatula. A metal split mold of 2 mm in width, 2 mm in height, and 25 mm in length was filled with the obtained mixed material. The bottom and top of the mold were each covered up by a metal plate, and pressure welding and fixation were carried out by a clamp. The clamped material was left in an atmosphere at a temperature of 37° C. and at a humidity of 100% for 1 hour to be cured. After that, the metal split mold was removed from the material. The obtained stick-shaped specimen was immersed in distilled water of 37° C. in temperature for 24 hours. Thereafter, the specimen was subjected to a compression test under a condition of 1 mm/min of crosshead speed, by a universal testing machine (Autograph, manufactured by Shimadzu Corporation). In each of the compositions according to Examples 1 to 8, any one of the fillers 1 to 6 was blended, and in each of the compositions according to Comparative Examples 1 to 3, any one of the fillers 9 to 11 was blended. No filler was blended in the composition according to Comparative Example 4. The results are together shown in Table 2.

TABLE 2
mass % is applied as unit, exclusive of “powder/liquid” and “Bending strength”

| | | | Example 1 | Example 2 | Example 3 | Example 4 | Example 5 | Example 6 | Example 7 |
|---|---|---|---|---|---|---|---|---|---|
| Powder | (A) Fluoroaluminosilicate | Fluoroalumino- | 91 | 91 | 95 | 93.5 | 92 | 93 | |
| component | glass powder | silicate glass | | | | | | | |
| | | powder A | | | | | | | |
| | | Fluoroalumino- | | | | | | | 95 |
| | | silicate glass | | | | | | | |
| | | powder B | | | | | | | |
| | (B) α-β unsaturated | Polyacrylic acid | 4 | 4 | 4 | 4 | 3 | 4 | 5 |
| | carboxylic acid polymer | | | | | | | | |
| | (D) Filler in which | Filler 1 | 5 | | | | | | |
| | compound having | Filler 2 | | 5 | | | | | |
| | carboxyl group(s) is/are | Filler 3 | | | 1 | 2.5 | 5 | | |
| | bound to surface of | Filler 6 | | | | | | 3 | |
| | inorganic powder via | | | | | | | | |
| | silicon atom | | | | | | | | |
| | Other filler | Filler 9 | | | | | | | |
| Liquid component | (B) α-β unsaturated | Polyacrylic acid | 45 | 45 | 45 | 45 | 45 | 45 | 43 |
| | carboxylic acid polymer | | | | | | | | |
| | (C) Water | | 50 | 50 | 50 | 50 | 50 | 50 | 45 |
| | (D) Filler in which | Filler 4 | | | | | | | 3 |
| | compound having | Filler 5 | | | | | | | |
| | carboxyl group(s) is/are | | | | | | | | |
| | bound to surface of | | | | | | | | |
| | inorganic powder via | | | | | | | | |
| | silicon atom | | | | | | | | |
| | Other filler | Filler 10 | | | | | | | |
| | | Filler 11 | | | | | | | |
| | (E) | Citric acid | 5 | 5 | 5 | 5 | 5 | 5 | 7 |
| | Acid (for adjusting pH) | | | | | | | | |

| | | | | | | | |
|---|---|---|---|---|---|---|---|
| powder/liquid (mass ratio) | 3.1 | 3.1 | 3.1 | 3.1 | 3.1 | 3.1 | 3.2 |
| Bending strength (MPa) | 32.0 | 35.1 | 36.8 | 40.6 | 43.1 | 42.0 | 40.2 |

| | | | | Comparative | Comparative | Comparative | Comparative |
|---|---|---|---|---|---|---|---|
| | | | Example 8 | Example 1 | Example 2 | Example 3 | Example 4 |
| Powder | (A) Fluoroaluminosilicate | Fluoroalumino- | | 93 | | | |
| component | glass powder | silicate glass | | | | | |
| | | powder A | | | | | |
| | | Fluoroalumino- | 95 | | 95 | 95 | 95 |
| | | silicate glass | | | | | |
| | | powder B | | | | | |
| | (B) α-β unsaturated | Polyacrylic acid | 5 | 2 | 5 | 5 | 5 |
| | carboxylic acid polymer | | | | | | |
| | (D) Filler in which | Filler 1 | | | | | |
| | compound having | Filler 2 | | | | | |
| | carboxyl group(s) is/are | Filler 3 | | | | | |
| | bound to surface of | Filler 6 | | | | | |
| | inorganic powder via | | | | | | |
| | silicon atom | | | | | | |
| | Other filler | Filler 9 | | 5 | | | |
| Liquid component | (B) α-β unsaturated | Polyacrylic acid | 43 | 43 | 43 | 43 | 43 |
| | carboxylic acid polymer | | | | | | |

| (C) Water | | 45 | 50 | 45 | 45 | 50 |
|---|---|---|---|---|---|---|
| (D) Filler in which | Filler 4 | | | | | |
| compound having | Filler 5 | 3 | | | | |
| carboxyl group(s) is/are | | | | | | |
| bound to surface of | | | | | | |
| inorganic powder via | | | | | | |
| silicon atom | | | | | | |
| Other filler | Filler 10 | | | 5 | | |
| | Filler 11 | | | | 5 | |
| (E) | Citric acid | 7 | 7 | 7 | 7 | 7 |
| Acid (for adjusting pH) | | | | | | |

| | | | | | |
|---|---|---|---|---|---|
| powder/liquid (mass ratio) | 3.3 | 3.3 | 3.4 | 3.4 | 3.4 |
| Bending strength (MPa) | 36.0 | 28.1 | 29.0 | 27.1 | 29.0 |

<<Making of Paste Type Dental Glass Ionomer Cement Composition>>

Each component was mixed at the blending amount shown in Table 3, whereby first pastes and second pastes of paste type dental glass ionomer cement compositions were prepared.

<<Pressure Resistance Strength Test>>

Each of the first pastes and second pastes prepared above was scaled at the paste ratios shown in Table 3, and mixed for 10 seconds on a mixing paper by a spatula. A metal split mold of 4 mm in diameter and 6 mm in height was filled with the obtained mixed material. The bottom and top of the mold were covered up by a metal plate. With a clamp, the mixed material with the mold was welded by pressure and fixed. The obtained material was left in an atmosphere at a temperature of 37° C. and at a humidity of 100% for 1 hour to be cured. After that, the metal split mold was removed from the material. The obtained cylindrical hardened cement was immersed in water of 37° C. in temperature for 24 hours. Thereafter, the specimen was subjected to a compression test under a condition of 1 mm/min of crosshead speed, by a universal testing machine (Autograph, manufactured by Shimadzu Corporation). In each of the compositions according to Examples 9 to 16, any one of the filler 1 to 4, 7 and 8 was blended, and in each of the compositions according to Comparative Examples 5 to 8, any one of the fillers 9 to 11 was blended. No filler was blended in the composition according to Comparative Example 9. The results are together shown in Table 3.

TABLE 3
mass % is applied as unit, exclusive of “first paste/second paste” and “Pressure resistance strength”

| | | | | | Example | Example | Example | Example | Example |
|---|---|---|---|---|---|---|---|---|---|
| | | | Example 9 | Example 10 | 11 | 12 | 13 | 14 | 15 |
| First | (A) | Fluoroaluminosilicate glass | 70 | 70 | 70 | 70 | 71 | 71 | |
| paste | Fluoroaluminosilicate | powder A | | | | | | | |
| | glass powder | Fluoroaluminosilicate glass | | | | | | | 50 |
| | | powder B | | | | | | | |
| | (C) Water | | 25 | 26 | 25 | 20 | 25 | 21 | 35 |
| | (F) Glycerin | | 4.5 | 3.5 | 4.5 | 9.5 | 3.6 | 7.6 | 4.8 |
| | (D) Filler in which | Filler 7 | | | | | | | 10 |
| | compound having | Filler 8 | | | | | | | |
| | carboxyl group(s) | | | | | | | | |
| | is/are bound to | | | | | | | | |
| | surface of inorganic | | | | | | | | |
| | powder via silicon atom | | | | | | | | |
| | (G) Thickening agent | Sodium carboxymethyl | 0.1 | 0.1 | 0.1 | 0.1 | | | |
| | | cellulose | | | | | | | |
| | | Fine particle silica | 0.4 | 0.4 | 04 | 0.4 | 0.4 | 0.4 | 0.2 |
| Second | (B) α-β unsaturated | Polyacrylic acid | 40 | 35 | 30 | 42 | 40 | 40 | 40 |
| paste | carboxylic acid | | | | | | | | |
| | polymer | | | | | | | | |
| | (C) Water | | 40 | 35 | 40 | 38 | 35 | 40 | 40 |
| | (D) Filler in which | Filler 1 | 20 | 30 | | | | | |
| | compound having | Filler 2 | | | 30 | 20 | | | |
| | carboxyl group(s) | Filler 3 | | | | | 25 | 20 | |
| | is/are bound to | Filler 4 | | | | | | | 20 |
| | surface of inorganic | | | | | | | | |
| | powder via silicon atom | | | | | | | | |
| | Other Filler | Filler 9 | | | | | | | |
| | | Filler 10 | | | | | | | |
| | | Filler 11 | | | | | | | |

| | | | | | | | |
|---|---|---|---|---|---|---|---|
| first paste/second paste (mass ratio) | 1.25 | 1.25 | 1.25 | 1.25 | 1.25 | 1.25 | 1 |
| Pressure resistance strength (MPa) | 43.1 | 44.2 | 48.0 | 46.3 | 56.2 | 55.3 | 57.5 |

| | | | | Comparative | Comparative | Comparative | Comparative | Comparative |
|---|---|---|---|---|---|---|---|---|
| | | | Example 16 | Example 5 | Example 6 | Example 7 | Example 8 | Example 9 |
| First | (A) | Fluoroaluminosilicate glass | | 64 | 64 | 64 | 64 | 64 |
| paste | Fluoroaluminosilicate | powder A | | | | | | |
| | glass powder | Fluoroaluminosilicate glass | 50 | | | | | |
| | | powder B | | | | | | |
| | (C) Water | | 37 | 31 | 30 | 33 | 33 | 31 |
| | (F) Glycerin | | 2.8 | 3.8 | 4.8 | 2.8 | 2.8 | 2.8 |
| | (D) Filler in which | Filler 7 | | | | | | |
| | compound having | Filler 8 | 10 | | | | | |
| | carboxyl group(s) | | | | | | | |
| | is/are bound to | | | | | | | |
| | surface of inorganic | | | | | | | |
| | powder via silicon atom | | | | | | | |
| | (G) Thickening agent | Sodium carboxymethyl | | 1 | 1 | | | 1 |
| | | cellulose | | | | | | |
| | | Fine particle silica | 0.2 | 0.2 | 0.2 | 0.2 | 0.2 | 0.2 |
| Second | (B) α-β unsaturated | Polyacrylic acid | 40 | 40 | 40 | 40 | 40 | 50 |
| paste | carboxylic acid | | | | | | | |
| | polymer | | | | | | | |
| | (C) Water | | 40 | 40 | 40 | 45 | 40 | 50 |
| | (D) Filler in which | Filler 1 | | | | | | |
| | compound having | Filler 2 | | | | | | |
| | carboxyl group(s) | Filler 3 | | | | | | |
| | is/are bound to | Filler 4 | 20 | | | | | |
| | surface of inorganic | | | | | | | |
| | powder via silicon atom | | | | | | | |
| | Other Filler | Filler 9 | | 20 | 20 | | | |
| | | Filler 10 | | | | 15 | | |
| | | Filler 11 | | | | | 20 | |

| | | | | | | |
|---|---|---|---|---|---|---|
| first paste/second paste (mass ratio) | 1 | 1 | 1 | 1 | 1 | 1 |
| Pressure resistance strength (MPa) | 54.0 | 39.5 | 36.9 | 38.9 | 40.7 | 30.3 |

From the results shown in Tables 2 and 3, it can be figured out that the dental glass ionomer cement composition according to the present invention had a higher strength when hardened, in both cases of a powder-paste type and a paste type, compared to the compositions of Comparative Examples.

1. A dental glass ionomer cement composition comprising a filler in which a compound(s) having a carboxyl group(s) is/are bound to a surface of an inorganic powder via a silicon atom, the dental glass ionomer cement composition not comprising a (meth)acrylate monomer.

2. A dental glass ionomer cement composition comprising:
a powder component whose main component is
(A) a fluoroaluminosilicate glass powder; and
a liquid component whose main components are
(B) an α-β unsaturated carboxylic acid polymer and
(C) water,
wherein:
at least one of the powder component and the liquid component include
(D) a filler in which a compound(s) having a carboxyl group(s) is/are bound to a surface of an inorganic powder via a silicon atom; and
neither the powder component nor the liquid component includes a (meth)acrylate monomer.

3. A dental glass ionomer cement composition comprising:
a first paste whose main components are
(A) a fluoroaluminosilicate glass powder and
(C) water; and
a second paste whose main components are
(B) an α-β unsaturated carboxylic acid polymer and
(C) water,
wherein:
the second paste includes
(D) a filler in which a compound(s) having a carboxyl group(s) is/are bound to a surface of an inorganic powder via a silicon atom; and
neither the first paste nor the second paste includes a (meth)acrylate monomer.
